(19) United States
(12) Patent Application Publication
(10) Pub. No.: US 2026/0187885 A1
(43) Pub. Date: Jul. 2, 2026

(54) IMAGE PROCESSING DEVICE AND IMAGE PROCESSING METHOD

(71) Applicant: HAMAMATSU PHOTONICS K.K., Hamamatsu-shi, Shizuoka (JP)

(72) Inventors: Fumio HASHIMOTO, Hamamatsu-shi, Shizuoka (JP); Kibo OTE, Hamamatsu-shi, Shizuoka (JP)

(73) Assignee: HAMAMATSU PHOTONICS K.K., Hamamatsu-shi, Shizuoka (JP)

(21) Appl. No.: 18/867,110

(22) PCT Filed: May 22, 2023
(86) PCT No.: PCT/JP2023/018986

§ 371 Date: Nov. 19, 2024

(30) Foreign Application Priority Data
May 26, 2022 (JP) 2022-086191

(51) Int. Cl. G06T 12/20; A61B 6/03; G06T 7/00

(52) U.S. Cl. CPC G06T 12/20; A61B 6/032; A61B 6/037; G06T 7/0012; G06T 2207/10081; G06T 2207/10088; G06T 2207/10104; G06T 2207/20021; G06T 2207/20036; G06T 2207/20081; G06T 2207/20084; G06T 2207/30016; G06T 2210/41; G06T 2211/441

(57) ABSTRACT

A radiation tomography system includes a radiation tomography apparatus and an image processing apparatus. The image processing apparatus includes a sinogram creation unit, a CNN processing unit, a convolution integration unit, a forward projection calculation unit, and a CNN training unit. The forward projection calculation unit performs forward projection calculation on a three-dimensional output image to create calculated sinograms divided into K blocks. The CNN training unit evaluates an error between a measured sinogram and the calculated sinogram for each of the K blocks, and trains a CNN based on an error evaluation result for each of the K blocks.

### TECHNICAL FIELD

The present disclosure relates to an apparatus and a method for creating a three-dimensional tomographic image of a subject based on coincidence information collected by a radiation tomography apparatus.

### BACKGROUND ART

Examples of a radiation tomography apparatus capable of acquiring a tomographic image of a subject (living body) include a positron emission tomography (PET) apparatus and a single photon emission computed tomography (SPECT) apparatus.

The PET apparatus includes a detection unit having a large number of small radiation detectors arranged around a measurement space in which the subject is placed. The PET apparatus detects a photon pair of an energy of 511 keV generated by electron positron annihilation in the subject into which a positron-emitting isotope (RI source) is injected by a coincidence method using the detection unit, and collects coincidence information of the detection.

Further, a tomographic image representing a spatial distribution of a generation frequency of the photon pairs in the measurement space (that is, a spatial distribution of the RI sources) can be reconstructed based on the collected many pieces of the coincidence information described above. The above PET apparatus plays an important role in a nuclear medicine field and the like, and can be used to study, for example, a biological function or a brain high-order function.

As a method for reconstructing the tomographic image of the subject based on the collected many pieces of the coincidence information, various methods are known. In an image processing method used for reconstructing the tomographic image which is described in Non Patent Document 1, the tomographic image is reconstructed by a deep image prior technique using a convolutional neural network, which is a type of a deep neural network. Hereinafter, the convolutional neural network is referred to as a “CNN”, and the deep image prior technique is referred to as a “DIP technique”.

The DIP technique takes advantage of the property of the CNN that meaningful structures in an image are learned faster than random noise (that is, the random noise is less likely to be learned). By using the DIP technique, the tomographic image in which the noise is reduced can be acquired.

Specifically, the image processing method described in Non Patent Document 1 is as follows. A sinogram (hereinafter, referred to as a “measured sinogram”) is created based on the many pieces of the coincidence information collected for the subject. Further, a sinogram (hereinafter, referred to as a “calculated sinogram”) is created by performing forward projection calculation (Radon transform) on an image output from the CNN when an input image (for example, an MRI image) is input to the CNN.

Further, an error between the calculated sinogram and the measured sinogram is evaluated, and the CNN is trained based on the error evaluation result. By using the DIP technique, when the image output from the CNN, the creation of the calculated sinogram by the forward projection calculation, the evaluation of the error, and the training of the CNN are repeatedly performed, the calculated sinogram gradually approaches the measured sinogram, and the output image from the CNN approaches the tomographic image of the subject.

The above image processing method includes a process of performing the forward projection from the CNN output image to the calculated sinogram, and on the other hand, does not include a process of performing back projection from the measured sinogram to the tomographic image, and thus, it is possible to acquire the tomographic image in which the noise is further reduced.

The sinogram is expressed as a histogram representing a frequency (a generation frequency of coincidence events) at which the coincidence information is acquired in a space (a sinogram space) represented by four variables of r, θ, z, and δ. The variable r represents a radius direction position of a coincidence detection line (a line connecting two detectors which perform coincidence detection of the photon pair). The variable θ represents an azimuth angle of the coincidence detection line. The variable z represents a center axis direction position of a midpoint of the coincidence detection line. Further, the variable δ represents a polar angle of the coincidence detection line.

### CITATION LIST

### Non Patent Literature

Non Patent Document 1: F. Hashimoto, K. Ote and Y. Onishi, “PET Image Reconstruction Incorporating Deep Image Prior and a Forward Projection Model”, IEEE Transactions on Radiation and Plasma Medical Sciences, doi: 10.1109/TRPMS.2022.3161569, 2022

### SUMMARY OF INVENTION

### Technical Problem

In general, a graphics processing unit (GPU) is used in the processing using the CNN. The GPU is an operation processing device specialized for the image processing, and includes an operation unit and a random access memory (RAM) which are integrated on one semiconductor chip. Various types of data used in the operation processing by the operation unit of the GPU are required to be stored in the RAM of the GPU.

In the image processing method described in Non Patent Document 1, the data to be stored in the RAM of the GPU includes, for example, the CNN input image, the CNN output image, a weight coefficient representing a training state of the CNN, a feature map, the measured sinogram, the calculated sinogram, a parameter necessary for the forward projection calculation, and the like, and requires an enormous storage capacity.

However, the capacity of the RAM of the GPU is limited, and thus, in the image processing method described in Non Patent Document 1, the two-dimensional forward projection calculation can be performed, and on the other hand, the three-dimensional forward projection calculation cannot be performed.

An object of the present invention is to provide an image processing apparatus and an image processing method capable of performing three-dimensional forward projection calculation from a CNN output image to a calculated sinogram, and easily creating a three-dimensional tomographic image of a subject by training a CNN based on an evaluation result of an error between the calculated sinogram and a measured sinogram.

### Solution to Problem

An embodiment of the present invention is an image processing apparatus. The image processing apparatus is an image processing apparatus for creating a three-dimensional tomographic image of a subject based on coincidence information collected by a radiation tomography apparatus including a plurality of detectors arranged around a measurement space in which the subject into which an RI source is injected is placed, and includes (1) a sinogram creation unit for creating a sinogram divided into a plurality of blocks based on the coincidence information collected by the radiation tomography apparatus; (2) a CNN processing unit for inputting a three-dimensional input image to a convolutional neural network, and creating a three-dimensional output image by the convolutional neural network; (3) a forward projection calculation unit for performing forward projection calculation on the three-dimensional output image to create a sinogram divided into the plurality of blocks; and (4) a CNN training unit for evaluating an error between the sinogram created by the sinogram creation unit and the sinogram created by the forward projection calculation unit for each of the plurality of blocks, and training the convolutional neural network based on an error evaluation result for each of the plurality of blocks, and the three-dimensional output image after respective processes of the CNN processing unit, the forward projection calculation unit, and the CNN training unit are repeatedly performed a plurality of times is set as the three-dimensional tomographic image of the subject.

An embodiment of the present invention is a radiation tomography system. The radiation tomography system includes a radiation tomography apparatus including a plurality of detectors arranged around a measurement space in which a subject into which an RI source is injected is placed, and for collecting coincidence information; and the image processing apparatus of the above configuration for creating the three-dimensional tomographic image of the subject based on the coincidence information collected by the radiation tomography apparatus.

An embodiment of the present invention is an image processing method. The image processing method is an image processing method for creating a three-dimensional tomographic image of a subject based on coincidence information collected by a radiation tomography apparatus including a plurality of detectors arranged around a measurement space in which the subject into which an RI source is injected is placed, and includes (1) a sinogram creation step of creating a sinogram divided into a plurality of blocks based on the coincidence information collected by the radiation tomography apparatus; (2) a CNN processing step of inputting a three-dimensional input image to a convolutional neural network, and creating a three-dimensional output image by the convolutional neural network; (3) a forward projection calculation step of performing forward projection calculation on the three-dimensional output image to create a sinogram divided into the plurality of blocks; and (4) a CNN training step of evaluating an error between the sinogram created in the sinogram creation step and the sinogram created in the forward projection calculation step for each of the plurality of blocks, and training the convolutional neural network based on an error evaluation result for each of the plurality of blocks, and the three-dimensional output image after respective processes of the CNN processing step, the forward projection calculation step, and the CNN training step are repeatedly performed a plurality of times is used as the three-dimensional tomographic image of the subject.

### Advantageous Effects of Invention

According to the embodiments of the present invention, it is possible to perform three-dimensional forward projection calculation from a CNN output image to a calculated sinogram, and easily create a three-dimensional tomographic image of a subject by training a CNN based on an evaluation result of an error between the calculated sinogram and a measured sinogram.

### BRIEF DESCRIPTION OF DRAWINGS

FIG. 1 is a diagram illustrating a configuration of a radiation tomography system 1.

FIG. 2 is a diagram illustrating a configuration example of a CNN.

FIG. 3 is a flowchart illustrating an image processing method.

FIG. 4 includes diagrams showing respective examples of a calculated sinogram 24 in the case of a comparative example and calculated sinograms 24₁ to 24₁₆ in the case of the present embodiment in comparison with each other, and shows (a) a diagram schematically showing the calculated sinogram 24 in the case of the comparative example, and (b) a diagram schematically showing the calculated sinograms 24₁ to 24₁₆ in the case of the present embodiment.

FIG. 5 includes (a)-(c) diagrams each showing a tomographic image of a brain obtained in a simulation.

FIG. 6 includes (a)-(c) diagrams each showing the tomographic image of the brain obtained in the simulation.

FIG. 7 includes (a)-(c) diagrams each showing the tomographic image of the brain obtained in the simulation.

FIG. 8 includes (a)-(c) diagrams each showing the tomographic image of the brain obtained in the simulation.

### DESCRIPTION OF EMBODIMENTS

Hereinafter, embodiments of an image processing apparatus and an image processing method will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

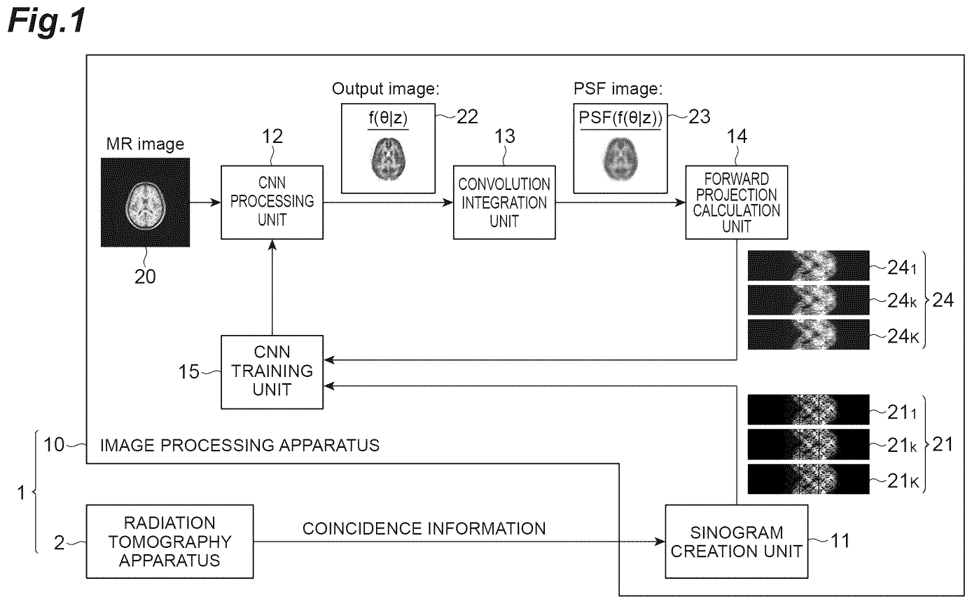

FIG. 1 is a diagram illustrating a configuration of a radiation tomography system 1. The radiation tomography system 1 includes a radiation tomography apparatus 2 and an image processing apparatus 10. The image processing apparatus 10 includes a sinogram creation unit 11, a CNN processing unit 12, a convolution integration unit 13, a forward projection calculation unit 14, and a CNN training unit 15.

The radiation tomography apparatus 2 is an apparatus for collecting coincidence information for reconstructing a tomographic image of a subject. Examples of the radiation tomography apparatus 2 include a PET apparatus and a SPECT apparatus. In the following description, it is assumed that the radiation tomography apparatus 2 is the PET apparatus.

The radiation tomography apparatus 2 includes a detection unit having a large number of small radiation detectors which are arranged around a measurement space in which the subject is placed. The radiation tomography apparatus 2 detects a photon pair of an energy of 511 keV generated by the electron positron annihilation in the subject into which an RI source is injected by a coincidence method by using the detection unit, and collects the coincidence information of the detection. Further, the radiation tomography apparatus 2 outputs the collected coincidence information to the image processing apparatus 10.

The image processing apparatus 10 includes a GPU for performing processing by using a CNN, an input unit (for example, a keyboard or a mouse) for receiving an input from an operator, a display unit (for example, a liquid crystal display) for displaying an image and the like, and a storage unit for storing a program and data for executing various types of the processing. As the image processing apparatus 10, a computer including a CPU, a RAM, a ROM, a hard disk drive, and the like is used.

The sinogram creation unit 11 creates a measured sinogram 21 based on the coincidence information collected by the radiation tomography apparatus 2. In this case, the sinogram creation unit 11 creates measured sinograms 21₁ to 21_{K} divided into a plurality of (K) blocks. The measured sinogram 21ₖ is a measured sinogram of the k-th block out of the K blocks. K is an integer of 2 or more, and k is an integer of 1 or more and K or less. The entire measured sinogram 21 is obtained by combining the divided measured sinograms 21₁ to 21_{K}.

The CNN processing unit 12 inputs a three-dimensional input image 20 to the CNN, and creates a three-dimensional output image 22 by the CNN. The three-dimensional input image 20 may be an image representing morphological information of the subject, may be an MRI image, a CT image, or a static PET image of the subject, or may be a random noise image.

The convolution integration unit 13 performs convolution integration of a point spread function on the three-dimensional output image 22 created by the CNN processing unit 12, and creates a new three-dimensional output image 23. The point spread function (PSF) is a function representing a response (impulse response) of the radiation tomography apparatus with respect to a point source, and is in general represented by a Gaussian function, an asymmetric Gaussian function in which the degree of blurring differs depending on a position in a visual field modeled from the measured data of the point source, or the like. By providing the convolution integration unit 13, the tomographic image with more excellent image quality can be obtained, and further, training of the CNN can be stabilized.

The forward projection calculation unit 14 performs forward projection calculation on the three-dimensional output image 23 to create a calculated sinogram 24. In this case, the forward projection calculation unit 14 creates calculated sinograms 24₁ to 24_{K} divided into the K blocks. The calculated sinogram 24ₖ is a calculated sinogram of the k-th block out of the K blocks. The entire calculated sinogram 24 is obtained by combining the divided calculated sinograms 24₁ to 24_{K}.

The block division of the calculated sinogram 24 is performed in the same manner as the block division of the measured sinogram 21. The calculated sinogram 24ₖ of the k-th block and the measured sinogram 21ₖ of the k-th block are the sinograms of a common region in an entire sinogram space. The configuration of the block division is set arbitrary, and the block division may be performed for any one or two or more variables out of the four variables representing the sinogram space. A size of each of the K blocks may be different, or may be the same.

The CNN training unit 15 evaluates an error between the measured sinogram 21ₖ and the calculated sinogram 24ₖ for each of the K blocks, and trains the CNN based on the error evaluation result for each of the K blocks.

The three-dimensional output image 22 which is created by the CNN processing unit 12 after the respective processes of the CNN processing unit 12, the convolution integration unit 13, the forward projection calculation unit 14, and the CNN training unit 15 are repeatedly performed a plurality of times is set as a three-dimensional tomographic image of the subject. The three-dimensional output image 23 which is created by the convolution integration unit 13 may be set as the three-dimensional tomographic image of the subject. The measured sinogram 21 is a sinogram reflecting the response function of the radiation tomography apparatus, and thus, the three-dimensional output image 22 before performing the convolution integration of the point spread function by the convolution integration unit 13 is preferably set as the three-dimensional tomographic image of the subject.

In addition, the convolution integration unit 13 may be provided as the final layer of the CNN, or may be provided separately from the CNN. In the case in which the convolution integration unit 13 is provided as the final layer of the CNN, a weight coefficient of the convolution integration unit 13 is maintained constant in the training of the CNN. Further, the convolution integration unit 13 may not be provided. In the case in which the convolution integration unit 13 is not provided, the forward projection calculation unit 14 creates the calculated sinogram 24 by performing the forward projection calculation on the three-dimensional output image 22 which is output from the CNN processing unit 12.

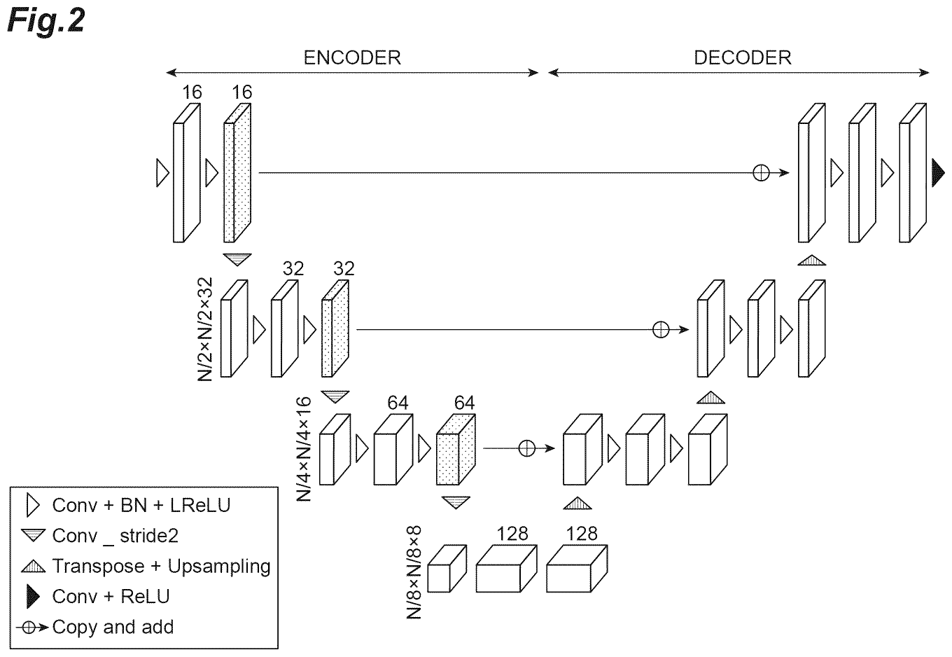

FIG. 2 is a diagram illustrating a configuration example of the CNN. The CNN illustrated in this diagram has a three-dimensional U-net structure including an encoder and a decoder. In this diagram, a size of each of the layers of the CNN is illustrated on the assumption that the number of pixels of the three-dimensional input image 20 which is input to the CNN is N×N×64.

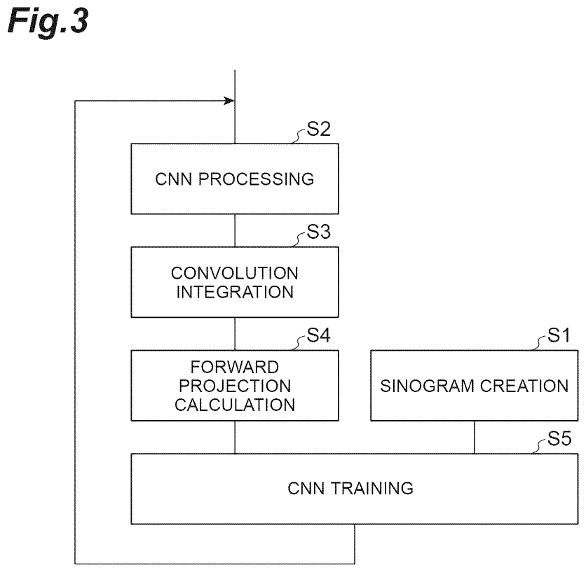

FIG. 3 is a flowchart of an image processing method. The image processing method includes a sinogram creation step S1 performed by the sinogram creation unit 11, a CNN processing step S2 performed by the CNN processing unit 12, a convolution integration step S3 performed by the convolution integration unit 13, a forward projection calculation step S4 performed by the forward projection calculation unit 14, and a CNN training step S5 performed by the CNN training unit 15.

In the sinogram creation step S1, the measured sinograms 21₁ to 21_{K} which are divided into the K blocks are created based on the coincidence information collected by the radiation tomography apparatus 2. In the CNN processing step S2, the three-dimensional input image 20 is input to the CNN, and the three-dimensional output image 22 is created by the CNN. In the convolution integration step S3, the convolution integration of the point spread function is performed on the three-dimensional output image 22 which is created in the CNN processing step S2, and the new three-dimensional output image 23 is created.

In the forward projection calculation step S4, the forward projection calculation is performed on the three-dimensional output image 23 to create the calculated sinograms 24₁ to 24_{K} which are divided into the K blocks. In the CNN training step S5, the error between the measured sinogram 21ₖ and the calculated sinogram 24ₖ is evaluated for each of the K blocks, and the CNN is trained based on the error evaluation result for each of the K blocks.

The three-dimensional output image 22 which is created in the CNN processing step S2 after the respective processes of the CNN processing step S2, the convolution integration step S3, the forward projection calculation step S4, and the CNN training step S5 are repeatedly performed the plurality of times is set as the three-dimensional tomographic image of the subject. The three-dimensional output image 23 which is created in the convolution integration step S3 may be set as the three-dimensional tomographic image of the subject. In addition, the convolution integration step S3 may not be provided.

Next, prior to the detailed description of the processing content of each of the steps in the image processing method of the present embodiment, a processing content of each of steps in an image processing method of a comparative example will be described. In the image processing method in the case of the comparative example, each of the measured sinogram and the calculated sinogram is not divided into a plurality of blocks.

Hereinafter, the processing by the CNN is set to f, the three-dimensional input image 20 which is input to the CNN is set to z, and a weight coefficient parameter representing a training state of the CNN is set to θ. As the training of the CNN progresses, θ changes. In the case in which the three-dimensional input image z is input to the CNN with the weight coefficient of θ, the three-dimensional output image 22 which is output from the CNN is set to x. The three-dimensional output image x is represented by the following Formula (1). In the CNN processing step, the processing represented by the above Formula is performed to create the three-dimensional output image x.

$$\begin{matrix}[ Formula 1 ] & \\ x = f ( θ \vert z ) & ( 1 )\end{matrix}$$

In the convolution integration step, the convolution integration of the point spread function is performed on the three-dimensional output image x which is created in the CNN processing step, and the new three-dimensional output image x is created. In addition, in FIG. 1, the three-dimensional output image x after the convolution integration is performed is denoted by PSF(f(θ|z)).

In the forward projection calculation step, the forward projection calculation is performed on the three-dimensional output image x to create the calculated sinogram 24. The calculated sinogram 24 is set to y, and a projection matrix for performing the forward projection calculation (Radon transform) from the three-dimensional output image x to the calculated sinogram y is set to P. The projection matrix is also referred to as a system matrix or a detection probability. The processing performed in the forward projection calculation step is represented by the following Formula (2).

$$\begin{matrix}[ Formula 2 ] & \\ y = P x & ( 2 )\end{matrix}$$

In the CNN training step, the measured sinogram 21 is set to y₀, the error between the measured sinogram y₀ and the calculated sinogram y (the above Formula (2)) is evaluated, and the CNN is trained based on the error evaluation result. The processing performed in the CNN training step is represented by the following Formula (3). A constrained optimization problem represented by the above Formula is a problem of optimizing the CNN parameter θ such that a value of an error evaluation function E(y; y₀) becomes small, under the constraint that the three-dimensional output image x which is created by the CNN is the tomographic image of the subject.

$$\begin{matrix}[ Formula 3 ] & \\ min E ( y ; {y}_{0} ) & ( 3 )\end{matrix}$$

$$s . t . x = f ( θ \vert z )$$

The constrained optimization problem represented by the above Formula (3) can be rewritten as an unconstrained optimization problem represented by the following Formula (4). The error evaluation function E is set to an arbitrary function, and for example, a L1 norm, a L2 norm, a negative log likelihood in a Poisson distribution, or the like can be used. In the case in which the L2 norm is used as the error evaluation function, the above Formula (4) can be rewritten as the following Formula (5).

$$\begin{matrix}[ Formula 4 ] & \\ min E ( Pf ( θ \vert z ) - {y}_{0} ) & ( 4 )\end{matrix}$$

$$\begin{matrix}[ Formula 5 ] & \\ {θ}^{*} = {arg min}_{θ} ‖ ( Pf ( θ \vert z ) - {y}_{0} ) ‖ & ( 5 )\end{matrix}$$

$${x}^{*} = f ( {θ}^{*} \vert z )$$

When the arrangement of the plurality of detectors provided in the radiation tomography apparatus is considered, there may be a region in which the collection of the coincidence information is impossible in the sinogram space. In consideration of the above fact, an optimization problem represented by the following Formula (6) may be used in place of the optimization problem represented by the above Formula (5). In the above Formula (6), m is a binary mask function, and has a value of 1 in a region in which the collection of the coincidence information is possible in the sinogram space, and has a value of 0 in a region in which the collection of the coincidence information is impossible. The above Formula (6) is a formula to selectively evaluate the error in the region in the sinogram space in which the collection of the coincidence information is possible by taking a Hadamard product of an error (y−y₀) and the binary mask function m.

$$\begin{matrix}[ Formula 6 ] & \\ {θ}^{*} = {arg min}_{θ} ‖ ( Pf ( θ \vert z ) - {y}_{0} ) ⊙ m ‖ & ( 6 )\end{matrix}$$

By repeatedly performing the respective processes of the CNN processing step, the convolution integration step, the forward projection calculation step, and the CNN training step the plurality of times, and solving the above optimization problem for the CNN parameter θ, the calculated sinogram y approaches the measured sinogram y₀, and the three-dimensional output image x which is created by the CNN approaches the tomographic image of the subject.

Next, the processing content of each of the steps in the image processing method of the present embodiment will be described in detail. In the present embodiment, in the forward projection calculation step, the forward projection calculation is performed on the three-dimensional output image x to create the calculated sinograms 24₁ to 24_{K} which are divided into the K blocks. The calculated sinogram 24ₖ of the k-th block is set to yₖ, and the projection matrix for performing the forward projection calculation (Radon transform) from the three-dimensional output image x to the calculated sinogram yₖ is set to Pₖ. The processing performed in the forward projection calculation step is represented by the following Formula (7).

$$\begin{matrix}[ Formula 7 ] & \\ {y}_{k} = {P}_{A} x ( k = 1 , 2 , 3 , … , K ) & ( 7 )\end{matrix}$$

In the CNN training step, the measured sinogram 21ₖ of the k-th block is set to y₀ₖ, the error between the measured sinogram y₀ₖ and the calculated sinogram yₖ is evaluated for each of the K blocks, and the CNN is trained based on the error evaluation result for each of the K blocks.

The processing performed in the CNN training step is represented by an unconstrained optimization problem of the following Formula (8). In the case in which the L2 norm is used as the error evaluation function, the following Formula (8) can be rewritten as the following Formula (9). Further, in the case in which the error is selectively evaluated in the region in the sinogram space in which the collection of the coincidence information is possible, it is represented by an unconstrained optimization problem of the following Formula (10). mₖ is a binary mask function in the k-th block.

$$\begin{matrix}[ Formula 8 ] & \\ min E ( {P}_{k} f ( θ \vert z ) - {y}_{0 k} ) & ( 8 )\end{matrix}$$

$$\begin{matrix}[ Formula 9 ] & \\ {θ}^{*} = {arg min}_{θ} ‖ ( {P}_{k} f ( θ \vert z ) - {y}_{0 k} ) ‖ ( k = 1 , 2 , 3 , … , K ) & ( 9 )\end{matrix}$$

$${x}^{*} = f ( {θ}^{*} \vert z )$$

$$\begin{matrix}[ Formula 10 ] & \\ {θ}^{*} = {arg min}_{θ} ‖ ( {P}_{k} f ( θ \vert z ) - {y}_{0 k} ) ⊙ {m}_{k} ‖ ( k = 1 , 2 , 3 , … , K ) & ( 10 )\end{matrix}$$

By repeatedly performing the respective processes of the CNN processing step, the convolution integration step, the forward projection calculation step, and the CNN training step the plurality of times, and solving the above optimization problem for the CNN parameter θ, the calculated sinogram yₖ for each of the K blocks approaches the measured sinogram y₀ₖ, and the three-dimensional output image x which is created by the CNN approaches the tomographic image of the subject.

Next, the comparison between the comparative example and the present embodiment regarding the storage capacity required for storing the data in the RAM of the GPU will be described. In this case, the number of pixels of the three-dimensional output image which is created by the CNN is set to 128×128×64, and the number of pixels of the sinogram space is set to 128×128×64×19. In the image processing method according to the present embodiment, it is set to K=16, and the forward projection calculation is performed on the three-dimensional output image to create the calculated sinograms 24₁ to 24₁₆ which are equally divided into 16 blocks.

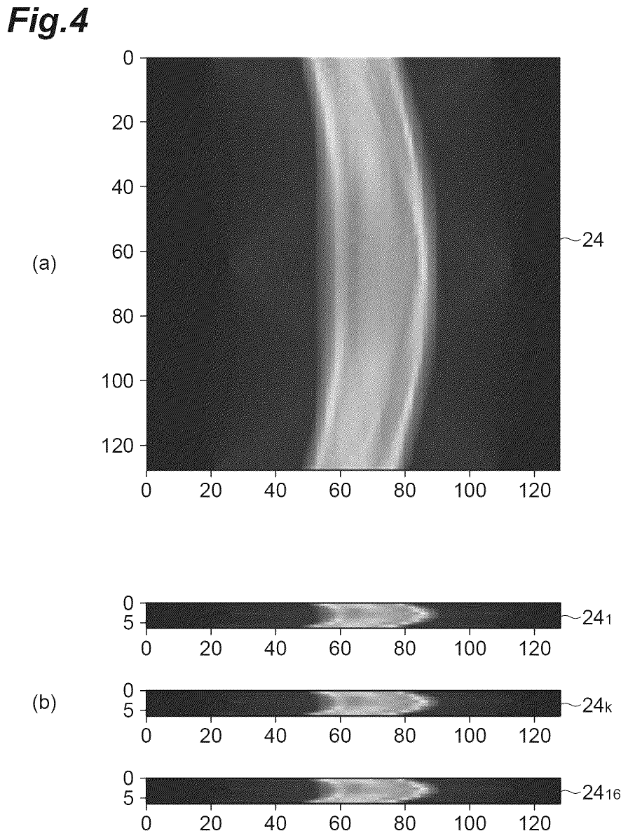

FIG. 4 includes diagrams showing respective examples of the calculated sinogram 24 in the case of the comparative example and the calculated sinograms 24₁ to 24₁₆ in the case of the present embodiment in comparison with each other. (a) in FIG. 4 is a diagram schematically showing the calculated sinogram 24 in the case of the comparative example. (b) in FIG. 4 is a diagram schematically showing the calculated sinograms 24₁ to 24₁₆ in the case of the present embodiment.

The number of pixels of the calculated sinogram 24ₖ of each of the blocks in the case of the present embodiment is 128×8×64×19, which is 1/16 of the number of pixels of the calculated sinogram 24 in the case of the comparative example. Further, the number of elements of the projection matrix Pₖ for performing the forward projection calculation from the three-dimensional output image to the calculated sinogram 24ₖ of the k-th block in the case of the present embodiment is 1/16 of the number of elements of the projection matrix P for performing the forward projection calculation from the three-dimensional output image to the calculated sinogram 24 in the case of the comparative example.

In the present embodiment, the storage capacity necessary for storing the data used at the time of performing the forward projection calculation can be reduced as compared with the case in the comparative example, and it is possible to store the above data in the RAM of the GPU. Therefore, in the present embodiment, it is possible to perform the three-dimensional forward projection calculation from the CNN output image to the calculated sinogram, and the three-dimensional tomographic image of the subject can be easily created by training the CNN based on the evaluation result of the error between the calculated sinogram and the measured sinogram.

Next, the result obtained by creating simulation data by using a Monte Carlo simulation of a head PET apparatus using a digital brain phantom image, and obtaining the tomographic image using the above data by each of the image processing method of the present embodiment and a ML-EM method will be described. The phantom image is obtained from BrainWeb (https://brainweb.bic.mni.mcgill.ca/brainweb/). The maximum likelihood expectation maximization (ML-EM) method is a general image reconstruction method.

In the image processing method of the present embodiment, the three-dimensional input image which is input to the CNN is set to the random noise image, the number of pixels of the input image is set to 128×128×64, the number of pixels of the three-dimensional output image which is created by the CNN is set to 128×128×64, the number of pixels of the sinogram space is set to 128×128×64×19, and the sinogram space is equally divided into the 16 blocks, and the error evaluation function is set to the negative log likelihood in the Poisson distribution. In the image processing method of the present embodiment, the number of repetitions is set to 2000, and further, in the ML-EM method, the number of repetitions is set to 50.

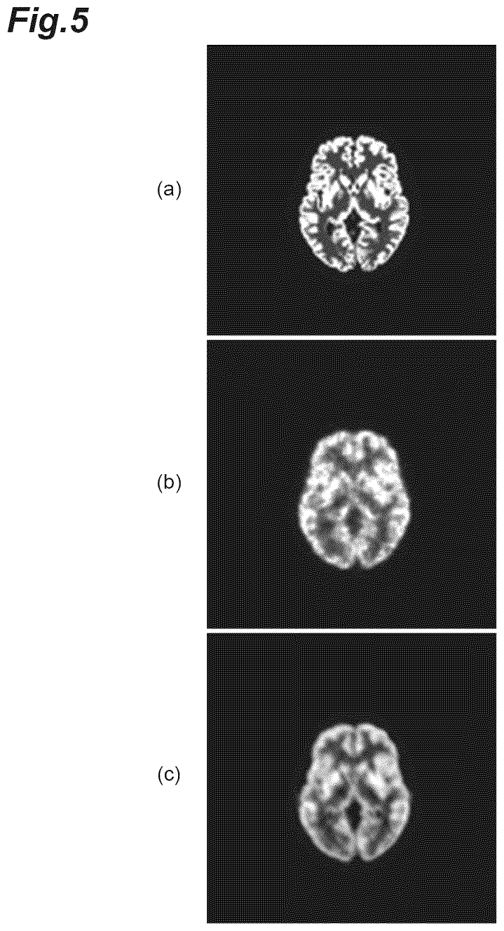

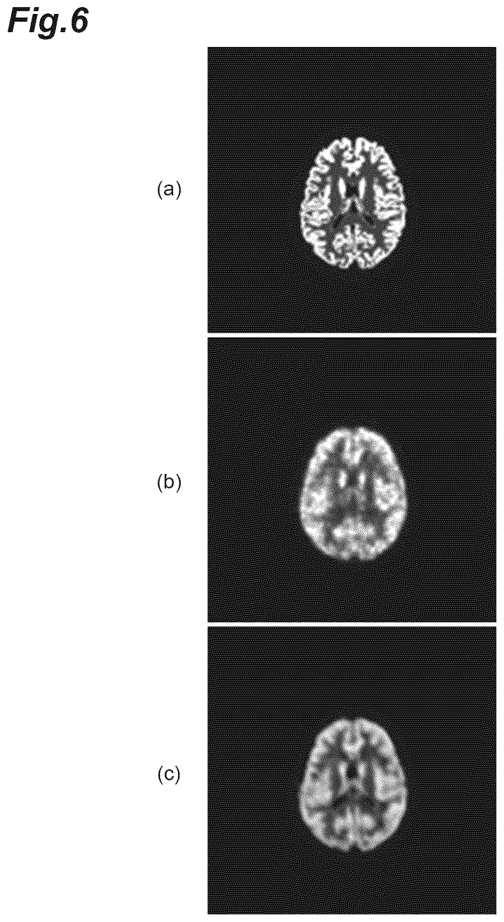

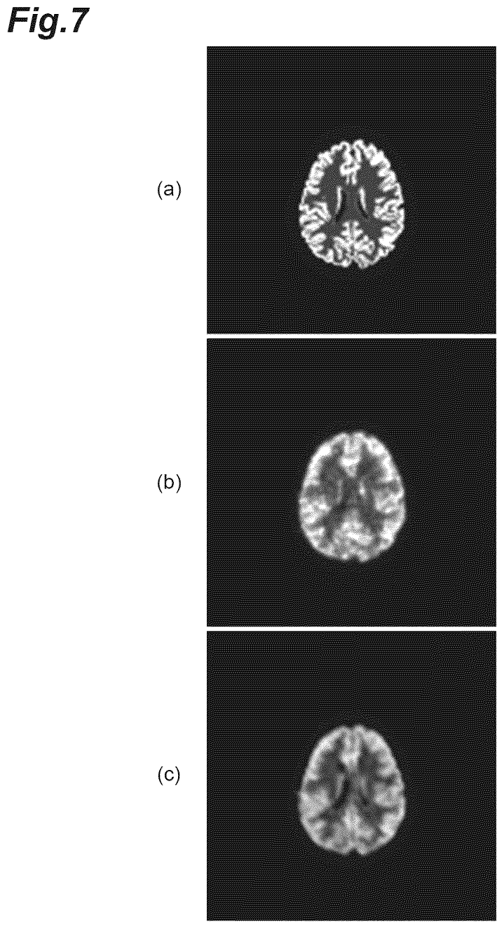

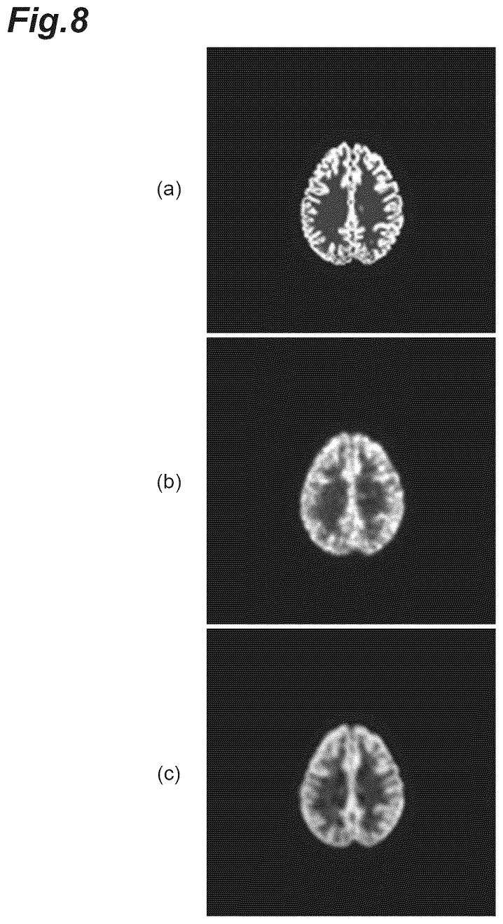

FIG. 5 to FIG. 8 include diagrams each showing the tomographic image of the brain obtained in the simulation. The above diagrams show the tomographic images of transverse sections respectively at four positions different from each other in a body axis direction in the three-dimensional tomographic image. In each of FIG. 5 to FIG. 8, (a) shows the phantom image (correct image), (b) shows the tomographic image which is obtained by the ML-EM method, and (c) shows the tomographic image which is obtained by the image processing method of the present embodiment.

In the image processing method of the present embodiment, the tomographic image with significantly excellent image quality can be obtained as compared with the case of the tomographic image obtained by the ML-EM method. Further, by the present simulation, it is confirmed that, in the present embodiment, the three-dimensional forward projection calculation from the CNN output image to the calculated sinogram can be performed by using the GPU, and the three-dimensional tomographic image of the subject can be created by training the CNN based on the evaluation result of the error between the calculated sinogram and the measured sinogram.

The image processing apparatus and the image processing method are not limited to the embodiments and configuration examples described above, and various modifications are possible.

The image processing apparatus of a first aspect according to the above embodiment is an image processing apparatus for creating a three-dimensional tomographic image of a subject based on coincidence information collected by a radiation tomography apparatus including a plurality of detectors arranged around a measurement space in which the subject into which an RI source is injected is placed, and includes (1) a sinogram creation unit for creating a sinogram divided into a plurality of blocks based on the coincidence information collected by the radiation tomography apparatus; (2) a CNN processing unit for inputting a three-dimensional input image to a convolutional neural network, and creating a three-dimensional output image by the convolutional neural network; (3) a forward projection calculation unit for performing forward projection calculation on the three-dimensional output image to create a sinogram divided into the plurality of blocks; and (4) a CNN training unit for evaluating an error between the sinogram created by the sinogram creation unit and the sinogram created by the forward projection calculation unit for each of the plurality of blocks, and training the convolutional neural network based on an error evaluation result for each of the plurality of blocks, and the three-dimensional output image after respective processes of the CNN processing unit, the forward projection calculation unit, and the CNN training unit are repeatedly performed a plurality of times is set as the three-dimensional tomographic image of the subject.

In the image processing apparatus of a second aspect, in the configuration of the first aspect, the apparatus may further include a convolution integration unit for performing convolution integration of a point spread function on the three-dimensional output image, and the forward projection calculation unit may perform the forward projection calculation on the three-dimensional output image after a process of the convolution integration unit is performed.

In the image processing apparatus of a third aspect, in the configuration of the first or second aspect, the CNN training unit may evaluate the error in a region in a sinogram space in which collection of the coincidence information by the radiation tomography apparatus is possible.

In the image processing apparatus of a fourth aspect, in the configuration of any one of the first to third aspects, the CNN processing unit may input an image representing morphological information of the subject to the convolutional neural network as the three-dimensional input image.

In the image processing apparatus of a fifth aspect, in the configuration of any one of the first to third aspects, the CNN processing unit may input an MRI image of the subject to the convolutional neural network as the three-dimensional input image.

In the image processing apparatus of a sixth aspect, in the configuration of any one of the first to third aspects, the CNN processing unit may input a CT image of the subject to the convolutional neural network as the three-dimensional input image.

In the image processing apparatus of a seventh aspect, in the configuration of any one of the first to third aspects, the CNN processing unit may input a static PET image of the subject to the convolutional neural network as the three-dimensional input image.

In the image processing apparatus of an eighth aspect, in the configuration of any one of the first to third aspects, the CNN processing unit may input a random noise image to the convolutional neural network as the three-dimensional input image.

The radiation tomography system according to the above embodiment includes a radiation tomography apparatus including a plurality of detectors arranged around a measurement space in which a subject into which an RI source is injected is placed, and for collecting coincidence information; and the image processing apparatus having the above configuration and for creating the three-dimensional tomographic image of the subject based on the coincidence information collected by the radiation tomography apparatus.

The image processing method of a first aspect according to the above embodiment is an image processing method for creating a three-dimensional tomographic image of a subject based on coincidence information collected by a radiation tomography apparatus including a plurality of detectors arranged around a measurement space in which the subject into which an RI source is injected is placed, and includes (1) a sinogram creation step of creating a sinogram divided into a plurality of blocks based on the coincidence information collected by the radiation tomography apparatus; (2) a CNN processing step of inputting a three-dimensional input image to a convolutional neural network, and creating a three-dimensional output image by the convolutional neural network; (3) a forward projection calculation step of performing forward projection calculation on the three-dimensional output image to create a sinogram divided into the plurality of blocks; and (4) a CNN training step of evaluating an error between the sinogram created in the sinogram creation step and the sinogram created in the forward projection calculation step for each of the plurality of blocks, and training the convolutional neural network based on an error evaluation result for each of the plurality of blocks, and the three-dimensional output image after respective processes of the CNN processing step, the forward projection calculation step, and the CNN training step are repeatedly performed a plurality of times is used as the three-dimensional tomographic image of the subject.

In the image processing method of a second aspect, in the configuration of the first aspect, the method may further include a convolution integration step of performing convolution integration of a point spread function on the three-dimensional output image, and in the forward projection calculation step, the forward projection calculation may be performed on the three-dimensional output image after a process of the convolution integration step is performed.

In the image processing method of a third aspect, in the configuration of the first or second aspect, in the CNN training step, the error may be evaluated in a region in a sinogram space in which collection of the coincidence information by the radiation tomography apparatus is possible.

In the image processing method of a fourth aspect, in the configuration of any one of the first to third aspects, in the CNN processing step, an image representing morphological information of the subject may be input to the convolutional neural network as the three-dimensional input image.

In the image processing method of a fifth aspect, in the configuration of any one of the first to third aspects, in the CNN processing step, an MRI image of the subject may be input to the convolutional neural network as the three-dimensional input image.

In the image processing method of a sixth aspect, in the configuration of any one of the first to third aspects, in the CNN processing step, a CT image of the subject may be input to the convolutional neural network as the three-dimensional input image.

In the image processing method of a seventh aspect, in the configuration of any one of the first to third aspects, in the CNN processing step, a static PET image of the subject may be input to the convolutional neural network as the three-dimensional input image.

In the image processing method of an eighth aspect, in the configuration of any one of the first to third aspects, in the CNN processing step, a random noise image may be input to the convolutional neural network as the three-dimensional input image.

### INDUSTRIAL APPLICABILITY

The present invention can be used as an image processing apparatus and an image processing method capable of performing three-dimensional forward projection calculation from a CNN output image to a calculated sinogram, and easily creating a three-dimensional tomographic image of a subject by training a CNN based on an evaluation result of an error between the calculated sinogram and a measured sinogram.

### REFERENCE SIGNS LIST

1—radiation tomography system, 2—radiation tomography apparatus, 10—image processing apparatus, 11—sinogram creation unit, 12—CNN processing unit, 13—convolution integration unit, 14—forward projection calculation unit, 15—CNN training unit.

1. An image processing apparatus for creating a three-dimensional tomographic image of a subject based on coincidence information collected by a radiation tomography apparatus including a plurality of detectors arranged around a measurement space in which the subject into which an RI source is injected is placed, the image processing apparatus comprising:
a sinogram creation unit configured to create a sinogram divided into a plurality of blocks based on the coincidence information collected by the radiation tomography apparatus;
a CNN processing unit configured to input a three-dimensional input image to a convolutional neural network, and create a three-dimensional output image by the convolutional neural network;
a forward projection calculation unit configured to perform forward projection calculation on the three-dimensional output image to create a sinogram divided into the plurality of blocks; and
a CNN training unit configured to evaluate an error between the sinogram created by the sinogram creation unit and the sinogram created by the forward projection calculation unit for each of the plurality of blocks, and train the convolutional neural network based on an error evaluation result for each of the plurality of blocks, wherein
the three-dimensional output image after respective processes of the CNN processing unit, the forward projection calculation unit, and the CNN training unit are repeatedly performed a plurality of times is set as the three-dimensional tomographic image of the subject.

2. The image processing apparatus according to claim 1, further comprising a convolution integration unit configured to perform convolution integration of a point spread function on the three-dimensional output image, wherein
the forward projection calculation unit is configured to perform the forward projection calculation on the three-dimensional output image after a process of the convolution integration unit is performed.

3. The image processing apparatus according to claim 1, wherein the CNN training unit is configured to evaluate the error in a region in a sinogram space in which collection of the coincidence information by the radiation tomography apparatus is possible.

4. The image processing apparatus according to claim 1, wherein the CNN processing unit is configured to input an image representing morphological information of the subject to the convolutional neural network as the three-dimensional input image.

5. The image processing apparatus according to claim 1, wherein the CNN processing unit is configured to input an MRI image of the subject to the convolutional neural network as the three-dimensional input image.

6. The image processing apparatus according to claim 1, wherein the CNN processing unit is configured to input a CT image of the subject to the convolutional neural network as the three-dimensional input image.

7. The image processing apparatus according to claim 1, wherein the CNN processing unit is configured to input a static PET image of the subject to the convolutional neural network as the three-dimensional input image.

8. The image processing apparatus according to claim 1, wherein the CNN processing unit is configured to input a random noise image to the convolutional neural network as the three-dimensional input image.

9. A radiation tomography system comprising:
a radiation tomography apparatus including a plurality of detectors arranged around a measurement space in which a subject into which an RI source is injected is placed, and configured to collect coincidence information; and
the image processing apparatus according to claim 1 configured to create the three-dimensional tomographic image of the subject based on the coincidence information collected by the radiation tomography apparatus.

10. An image processing method for creating a three-dimensional tomographic image of a subject based on coincidence information collected by a radiation tomography apparatus including a plurality of detectors arranged around a measurement space in which the subject into which an RI source is injected is placed, the image processing method comprising:
a sinogram creation step of creating a sinogram divided into a plurality of blocks based on the coincidence information collected by the radiation tomography apparatus;
a CNN processing step of inputting a three-dimensional input image to a convolutional neural network, and creating a three-dimensional output image by the convolutional neural network;
a forward projection calculation step of performing forward projection calculation on the three-dimensional output image to create a sinogram divided into the plurality of blocks; and
a CNN training step of evaluating an error between the sinogram created in the sinogram creation step and the sinogram created in the forward projection calculation step for each of the plurality of blocks, and training the convolutional neural network based on an error evaluation result for each of the plurality of blocks, wherein
the three-dimensional output image after respective processes of the CNN processing step, the forward projection calculation step, and the CNN training step are repeatedly performed a plurality of times is used as the three-dimensional tomographic image of the subject.

11. The image processing method according to claim 10, further comprising a convolution integration step of performing convolution integration of a point spread function on the three-dimensional output image, wherein
in the forward projection calculation step, the forward projection calculation is performed on the three-dimensional output image after a process of the convolution integration step is performed.

12. The image processing method according to claim 10, wherein in the CNN training step, the error is evaluated in a region in a sinogram space in which collection of the coincidence information by the radiation tomography apparatus is possible.

13. The image processing method according to claim 10, wherein in the CNN processing step, an image representing morphological information of the subject is input to the convolutional neural network as the three-dimensional input image.

14. The image processing method according to claim 10, wherein in the CNN processing step, an MRI image of the subject is input to the convolutional neural network as the three-dimensional input image.

15. The image processing method according to claim 10, wherein in the CNN processing step, a CT image of the subject is input to the convolutional neural network as the three-dimensional input image.

16. The image processing method according to claim 10, wherein in the CNN processing step, a static PET image of the subject is input to the convolutional neural network as the three-dimensional input image.

17. The image processing method according to claim 10, wherein in the CNN processing step, a random noise image is input to the convolutional neural network as the three-dimensional input image.
